SEQUENCE LISTING (30 sequences)

SEQ ID NO: 1 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 2 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 3 - protein, 170 aa, Homo sapiens
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 4 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 125 aa, Artificial Sequence
  Nanobody or nanobody construct
EVQLLESGGGLVQPGGSLRLSCAASGRIFSLPASGNIFNLLTIAWYRQAPGKGRELVSTINSGSRTYYADSVKGRFTISRDNSKKTLYLQMNSLRPEDTAVYYCQTSGSGSPNFWGQGTLVTVSS

SEQ ID NO: 6 - protein, 125 aa, Artificial Sequence
  Nanobody or nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSS

SEQ ID NO: 7 - protein, 125 aa, Artificial Sequence
  Nanobody or nanobody construct
EVQLLESGGGLVQPGGSLRLSCAASGRIFSLPASGNIFNLLTIAWYRQAPGKGRELVATIESGSRTYYADSVKGRFTISRDNSKKTVYLQMNSLRPEDTAVYYCQTSGSGSPNFWGQGTLVTVSS

SEQ ID NO: 8 - protein, 125 aa, Artificial Sequence
  Nanobody or nanobody construct
EVQLLESGGGLVQPGGSLRLSCAASGRIFSLPASGNIFNLLTIAWYRQAPGKGRELVATIESGSRTYYADSVKGRFTISRDNSKKTVYLQMNNLRPEDTAVYYCQTSGSGSPNFWGQGTLVTVSS

SEQ ID NO: 9 - protein, 125 aa, Artificial Sequence
  Nanobody or nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNNLRPEDTAVYYCQTSGSGSPNFWGQGTLVTVSS

SEQ ID NO: 10 - protein, 128 aa, Artificial Sequence
  Nanobody or nanobody construct
EVQLLESGGGLVQPGGSLRLSCAASGRTLSSYAMGWFRQAPGKGREFVARISQGGTAIYYADSVKGRFTISRDNSKNTLYLQMNSLRPEDTAVYYCAKDPSPYYRGSAYLLSGSYDSWGQGTLVTVSS

SEQ ID NO: 11 - protein, 128 aa, Artificial Sequence
  Nanobody or nanobody construct
EVQLLESGGGLVQPGGSLRLSCAASGRTLSSYAMGWFRQAPGKGREFVARISQGGTAIYYADSVKGRFTISRDNSKNTVYLQMNSLRPEDTAVYYCAKDPSPYYRGSAYLLSGSYDSWGQGTLVTVSS

SEQ ID NO: 12 - protein, 125 aa, Artificial Sequence
  Nanobody or nanobody construct
EVQLLESGGGLVQPGGSLRLSCAASGFTLDDYAIAWFRQAPGKGREGVSGIDSGDGSAYYADSVKGRFTISSDNSKNTVYLQMNSLRPEDTAVYYCARVRTGWGLNAPDYAMDYWGQGTLVTVSS

SEQ ID NO: 13 - protein, 125 aa, Artificial Sequence
  Nanobody or nanobody construct
EVQLLESGGGLVQPGGSLRLSCAASGFTLDDYAIAWFRQAPGKGREGVSGIDSGEGSAYYADSVKGRFTISSDNSKNTVYLQMNSLRPEDTAVYYCARVRTGWGLNAPDYAMDYWGQGTLVTVSS

SEQ ID NO: 14 - protein, 125 aa, Artificial Sequence
  Nanobody or nanobody construct
EVQLLESGGGLVQPGGSLRLSCAASGFTLDDYAIAWFRQAPGKGREGVSGIDAGEGSAYYADSVKGRFTISSDNSKNTVYLQMNSLRPEDTAVYYCARVRTGWGLNAPDYAMDYWGQGTLVTVSS

SEQ ID NO: 15 - protein, 126 aa, Artificial Sequence
  Nanobody or nanobody construct
EVQLLESGGGLVQPGGSLRLSCAASGFTLDYLAIGWFRQAPGKGREGVSCVSSSGQYTYYADSVKGRFTISRDNSESTVYLQMNSLRPEDTAVYYCATDPECYRVRGYYNAEYDYWGQGTLVTVSS

SEQ ID NO: 16 - protein, 386 aa, Artificial Sequence
  Nanobody or nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLLESGGGLVQPGGSLRLSCAASGRTLSSYAMGWFRQAPGKGREFVSRISQGGTAIYYADSVKGRFTISRDNSKNTLYLQMNSLRPEDTAVYYCAKDPSPYYRGSAYLLSGSYDSWGQGTLVTVSS

SEQ ID NO: 17 - protein, 386 aa, Artificial Sequence
  Nanobody or nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGSEVQLLESGGGLVQPGGSLRLSCAASGRTLSSYAMGWFRQAPGKGREFVSRISQGGTAIYYADSVKGRFTISRDNSKNTLYLQMNSLRPEDTAVYYCAKDPSPYYRGSAYLLSGSYDSWGQGTLVTVSS

SEQ ID NO: 18 - protein, 386 aa, Artificial Sequence
  Nanobody or nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLLESGGGLVQPGGSLRLSCAASGRTLSSYAMGWFRQAPGKGREFVARISQGGTAIYYADSVKGRFTISRDNSKNTLYLQMNSLRPEDTAVYYCAKDPSPYYRGSAYLLSGSYDSWGQGTLVTVSS

SEQ ID NO: 19 - protein, 386 aa, Artificial Sequence
  Nanobody or nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGSEVQLVESGGGLVQPGNSLRLSCAASGFTFRSFGMSWVRQAPGKEPEWVSSISGSGSDTLYADSVKGRFTISRDNAKTTLYLQMNSLKPEDTAVYYCTIGGSLSRSSQGTQVTVSSGGGGSGGGSEVQLVESGGGLVQAGGSLRLSCAASGRTLSSYAMGWFRQAPGKEREFVARISQGGTAIYYADSVKGRFTISRDNAKNTVYLQMNSLKPEDTAVYYCAKDPSPYYRGSAYLLSGSYDSWGQGTQVTVSS

SEQ ID NO: 20 - protein, 288 aa, Artificial Sequence
  Nanobody or nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGGSGGGGSGGGGSGGGGSGGGGSGGGGSEVQLVESGGGLVQAGGSLRLSCAASGRTLSSYAMGWFRQAPGKEREFVARISQGGTAIYYADSVKGRFTISRDNAKNTVYLQMNSLKPEDTAVYYCAKDPSPYYRGSAYLLSGSYDSWGQGTQVTVSS

SEQ ID NO: 21 - protein, 398 aa, Artificial Sequence
  Nanobody or nanobody construct
EVQLLESGGGLVQPGGSLRLSCAASGRTLSSYAMGWFRQAPGKGREFVARISQGGTAIYYADSVKGRFTISRDNSKNTLYLQMNSLRPEDTAVYYCAKDPSPYYRGSAYLLSGSYDSWGQGTLVTVSSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSEVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSS

SEQ ID NO: 22 - protein, 398 aa, Artificial Sequence
  Nanobody or nanobody construct
EVQLLESGGGLVQPGGSLRLSCAASGRTLSSYAMGWFRQAPGKGREFVARISQGGTAIYYADSVKGRFTISRDNSKNTVYLQMNSLRPEDTAVYYCAKDPSPYYRGSAYLLSGSYDSWGQGTLVTVSSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSEVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSS

SEQ ID NO: 23 - protein, 386 aa, Artificial Sequence; identical to SEQ ID NO: 18
  Nanobody or nanobody construct

SEQ ID NO: 24 - protein, 386 aa, Artificial Sequence
  Nanobody or nanobody construct
EVQLLESGGGLVQPGGSLRLSCAASGRIFSLPASGNIFNLLTIAWYRQAPGKGRELVATINSGSRTYYADSVKGRFTISRDNSKKTVYLQMNSLRPEDTAVYYCQTSGSGSPNFWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLLESGGGLVQPGGSLRLSCAASGRTLSSYAMGWFRQAPGKGREFVARISQGGTAIYYADSVKGRFTISRDNSKNTVYLQMNSLRPEDTAVYYCAKDPSPYYRGSAYLLSGSYDSWGQGTLVTVSS

SEQ ID NO: 25 - protein, 398 aa, Artificial Sequence
  Nanobody or nanobody construct
EVQLLESGGGLVQPGGSLRLSCAASGRTLSSYAMGWFRQAPGKGREFVARISQGGTAIYYADSVKGRFTISRDNSKNTVYLQMNSLRPEDTAVYYCAKDPSPYYRGSAYLLSGSYDSWGQGTLVTVSSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSEVQLLESGGGLVQPGGSLRLSCAASGRIFSLPASGNIFNLLTIAWYRQAPGKGRELVATIESGSRTYYADSVKGRFTISRDNSKKTVYLQMNSLRPEDTAVYYCQTSGSGSPNFWGQGTLVTVSS

SEQ ID NO: 26 - protein, 398 aa, Artificial Sequence
  Nanobody or nanobody construct
EVQLLESGGGLVQPGGSLRLSCAASGRTLSSYAMGWFRQAPGKGREFVARISQGGTAIYYADSVKGRFTISRDNSKNTVYLQMNSLRPEDTAVYYCAKDPSPYYRGSAYLLSGSYDSWGQGTLVTVSSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSEVQLLESGGGLVQPGGSLRLSCAASGRIFSLPASGNIFNLLTIAWYRQAPGKGRELVATIESGSRTYYADSVKGRFTISRDNSKKTVYLQMNNLRPEDTAVYYCQTSGSGSPNFWGQGTLVTVSS

SEQ ID NO: 27 - protein, 398 aa, Artificial Sequence
  Nanobody or nanobody construct
EVQLLESGGGLVQPGGSLRLSCAASGRTLSSYAMGWFRQAPGKGREFVARISQGGTAIYYADSVKGRFTISRDNSKNTVYLQMNSLRPEDTAVYYCAKDPSPYYRGSAYLLSGSYDSWGQGTLVTVSSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGGSGGGGSEVQLLESGGGLVQPGGSLRLSCAASGRIFSLPASGNIFNLLTIAWYRQAPGKGRELVATINSGSRTYYADSVKGRFTISRDNSKKTVYLQMNNLRPEDTAVYYCQTSGSGSPNFWGQGTLVTVSS

SEQ ID NO: 28 - protein, 390 aa, Artificial Sequence
  Nanobody or nanobody construct
EVQLLESGGGLVQPGGSLRLSCAASGFTLDDYAIAWFRQAPGKGREGVSGIDSGDGSAYYADSVKGRFTISSDNSKNTVYLQMNSLRPEDTAVYYCARVRTGWGLNAPDYAMDYWGQGTLVTVSSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLLESGGGLVQPGGSLRLSCAASGFTLDYLAIGWFRQAPGKGREGVSCVSSSGQYTYYADSVKGRFTISRDNSESTVYLQMNSLRPEDTAVYYCATDPECYRVRGYYNAEYDYWGQGTLVTVSS

SEQ ID NO: 29 - protein, 390 aa, Artificial Sequence
  Nanobody or nanobody construct
EVQLLESGGGLVQPGGSLRLSCAASGFTLDDYAIAWFRQAPGKGREGVSGIDSGEGSAYYADSVKGRFTISSDNSKNTVYLQMNSLRPEDTAVYYCARVRTGWGLNAPDYAMDYWGQGTLVTVSSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLLESGGGLVQPGGSLRLSCAASGFTLDYLAIGWFRQAPGKGREGVSCVSSSGQYTYYADSVKGRFTISRDNSESTVYLQMNSLRPEDTAVYYCATDPECYRVRGYYNAEYDYWGQGTLVTVSS

SEQ ID NO: 30 - protein, 390 aa, Artificial Sequence
  Nanobody or nanobody construct
EVQLLESGGGLVQPGGSLRLSCAASGFTLDDYAIAWFRQAPGKGREGVSGIDAGEGSAYYADSVKGRFTISSDNSKNTVYLQMNSLRPEDTAVYYCARVRTGWGLNAPDYAMDYWGQGTLVTVSSGGGGSGGGGSGGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLLESGGGLVQPGGSLRLSCAASGFTLDYLAIGWFRQAPGKGREGVSCVSSSGQYTYYADSVKGRFTISRDNSESTVYLQMNSLRPEDTAVYYCATDPECYRVRGYYNAEYDYWGQGTLVTVSS

(19) United States
(12) Patent Application Publication
(10) Pub. No.: US 2012/0264917 A1
(43) Pub. Date: Oct. 18, 2012

(54) BIPARATOPIC PROTEIN CONSTRUCTS DIRECTED AGAINST IL-23

(75) Inventors: Michael John Scott Saunders, Brussels (BE); Christophe Blanchetot, Destelbergen (BE); Carlo Boutton, Wielsbeke (BE); Heidi Rommelaere, Gent (BE); Johannes Joseph Wilhelmus De Haard, Oudelande (NL); Veronique De Brabandere, Gent (BE); Marc Jozef Lauwereys, Haaltert (BE); Erika Morizzo, Gent (BE); Ann Union, Aalter (BE); Gert Verheyden, Bavegem (BE)

(73) Assignee: Ablynx N.V., Zwijnaarde (BE)

(21) Appl. No.: 13/321,979

(22) PCT Filed: May 27, 2010
(86) PCT No.: PCT/EP10/57341

§ 371 Date: Jul. 2, 2012

Related U.S. Application Data
(60) Provisional application No. 61/181,384, filed on May 27, 2009.

(51) Int. Cl. C07K 16/46

(52) U.S. Cl. 530/387.3

(57) ABSTRACT

Biparatopic protein constructs that are directed against IL-23, and in particular against the p19 subunit of IL-23. The constructs comprise at least a first binding domain or binding unit directed against a first defined epitope on p19 and at least a second binding domain or binding unit directed against a second defined epitope on p19 (or the p19/p40 interface). The binding domains or binding units may in particular be a domain antibody, a single domain antibody, a dAb or a Nanobody®. The constructs and pharmaceutical compositions comprising the same can be used for the prevention and/or treatment of diseases and disorders associated with IL-23 or IL-23 mediated signaling, such as inflammation and inflammatory disorders such as colitis, Crohn's disease and IBD, infectious diseases, psoriasis, cancer, autoimmune diseases, sarcoidosis, transplant rejection, cystic Fibrosis, asthma, chronic obstructive pulmonary disease, rheumatoid arthritis, viral infection, and common variable immunodeficiency.

The present invention relates to biparatopic proteins and polypeptides that are directed against IL-23 (also referred to interchangeably herein as “compounds of the invention”, “amino acid sequences of the invention”, or “constructs of the invention”).

The invention also relates to nucleic acids encoding the compounds of the invention (also referred to herein as “nucleic acids of the invention” or “nucleotide sequences of the invention”); to methods for preparing the compounds of the invention; to host cells expressing or capable of expressing the compounds of the invention; to compositions, and in particular to pharmaceutical compositions, that comprise the compounds of the invention; and to uses of the compounds of the invention and the aforementioned nucleic acids, host cells and/or compositions, in particular for prophylactic, therapeutic or diagnostic purposes, such as the prophylactic, therapeutic or diagnostic purposes mentioned herein.

Other aspects, embodiments, advantages and applications of the invention will become clear from the further description herein.

The International application PCT/EP2008/066365 of Ablynx N.V. (filed on Nov. 27, 2008 and entitled “Amino acid sequences directed against heterodimeric cytokines and/or their receptors and polypeptides comprising the same” and published on Jun. 4, 2009 as WO 09/068,627)) describes amino acid sequences (such as domain antibodies, single domain antibodies, dAbs, VHH's and Nanobodies®) that are directed against heterodimeric cytokines. Unless explicitly stated otherwise herein, all terms used in the present application are as defined in PCT/EP2008/066365. Also, the teaching of PCT/EP2008/066365 is incorporated herein by reference.

One aspect of PCT/EP2008/066365 relates to amino acid sequences that are directed against and specific for IL-23. For example, in one specific aspect, PCT/EP2008/066365 describes “multivalent” (as defined in PCT/EP2008/066365), “multispecific” (as defined in PCT/EP2008/066365) and in particular “biparatopic” (as defined in PCT/EP2008/066365) constructs that are directed against IL-23. Some non-limiting examples thereof are the biparatopic anti-p19 constructs described in Example 29, the biparatopic anti-p19 constructs described in Example 46, and the anti-p19/anti-p40 constructs that are also described in Example 46.

Applicant has now identified some particularly preferred classes of multispecific (and in particular bispecific) and multiparatopic (and in particular biparatopic) constructs that are directed against IL-23. In doing so, applicant has also identified some particularly preferred binding interactions and epitopes on IL-23 for (monovalent, multispecific and/or biparatopic) binders that bind to IL-23.

As generally described in PCT/EP2008/066365, the biparatopic constructs described herein generally comprise (at least) two binding domains, binding units or binding sites, of which at least one binding domain, binding unit or binding site is directed against a first epitope or antigenic determinant on IL-23, and at least one binding domain, binding unit or binding site is directed against a second epitope or antigenic determinant on IL-23 different from the first.

As further described in PCT/EP2008/066365, these at least two binding domains, binding units or binding sites are preferably suitably linked to each other, either directly (as generally described in PCT/EP2008/066365). or via one or more suitable spacers or linkers (again as generally described in PCT/EP2008/066365). In one specific aspect, the binding domains present in the compounds of the invention are both amino acid sequences (and in particular, “amino acid sequences of the invention” as generally described in PCT/EP2008/066365) which are linked to each other via a peptide linker (again as generally described in PCT/EP2008/066365), so that the resulting compound of the invention is a fusion protein or polypeptide.

Where the compounds of the invention comprise (at least two) distinct binding domains or binding units, these binding domains or binding units may generally “amino acid sequences of the invention” as described in PCT/EP2008/066365. In particular, as described in PCT/EP2008/066365, the binding domains may be amino acid sequences that comprise an immunoglobulin fold or may be amino acid sequences that, under suitable conditions (such as physiological conditions) are capable of forming an immunoglobulin fold (i.e. by folding). Reference is inter alia made to the review by Halaby et al., J. (1999) Protein Eng. 12, 563-71. For example, for this purpose, such amino acid sequences may be amino acid sequences that essentially consist of 4 framework regions (FR1 to FR4 respectively) and 3 complementarity determining regions (CDR1 to CDR3 respectively); or may be any suitable fragment of such an amino acid sequence (which will then usually contain at least some of the amino acid residues that form at least one of the CDR's, as further described herein). More in particular, the framework regions of such amino acid sequences may be as described in detail in PCT/EP2008/066365 (e.g. for the framework regions of Nanobodies®). Also, any such parts, fragments, analogs, mutants, variants, alleles and/or derivatives of such amino acid sequences are preferably such that they comprise an immunoglobulin fold or are capable for forming, under suitable conditions, an immunoglobulin fold.

More in particular, as further described in PCT/EP2008/066365, such amino acid sequences may be a domain antibody (or an amino acid sequence that is suitable for use as a domain antibody), a single domain antibody (or an amino acid sequence that is suitable for use as a single domain antibody), a “dAb” (or an amino acid sequence that is suitable for use as a dAb) or a Nanobody® (as further described in PCT/EP2008/066365, and including but not limited to a V_{HH} sequence, a humanized V_{HH} sequence, or an amino acid sequence that is characterized by the presence of one or more “Hallmark residues” as described in PCT/EP2008/066365 in one or more of the framework sequences, again as further described in PCT/EP2008/066365); other single variable domains, or any suitable fragment of any one thereof. For a general description of (single) domain antibodies, reference is also made to the prior art cited above, as well as to EP 0 368 684. For the term “dAb's”, reference is for example made to Ward et al. (Nature 1989 Oct. 12; 341 (6242): 544-6), to Holt et al., Trends Biotechnol., 2003, 21(11):484-490; as well as to for example WO 06/030220, WO 06/003388 and other published patent applications of Domantis Ltd. It should also be noted that, although less preferred in the context of the present invention because they are not of mammalian origin, single domain antibodies or single variable domains can be derived from certain species of shark (for example, the so-called “IgNAR domains”, see for example WO 05/18629).

In particular, the amino acid sequence of the invention may be a Nanobody® of the invention as described in PCT/EP2008/066365.

Preferably, the amino acid sequences used as binding domains or binding units in the compounds of the invention are preferably “directed against” and/or “specific for” (as defined in PCT/EP2008/066365) IL-23, and in particular for the subunit(s) of IL-23 against which they are directed. In particular, the amino acid sequences and polypeptides of the invention are preferably such that they:
bind to IL-23 (and in particular for the subunit(s) of IL-23 against which they are directed) with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles);
and/or such that they:
bind to IL-23 (and in particular for the subunit(s) of IL-23 against which they are directed) with a kₒₙ-rate of between 10² M⁻¹s⁻¹ to about 10⁷ M⁻¹s⁻¹, preferably between 10³ M⁻¹s⁻¹ and 10⁷ M⁻¹s⁻¹, more preferably between 10⁴ M⁻¹s⁻¹ and 10⁷M⁻¹s⁻¹, such as between 10⁵ M⁻¹s⁻¹ and 10⁷ M⁻¹s⁻¹; and/or such that they:
bind to IL-23 (and in particular for the subunit(s) of IL-23 against which they are directed) with a k_{off} rate between 1 s⁻¹ (t_{1/2}=0.69 s) and 10⁻⁶ s⁻¹ (providing a near irreversible complex with a t_{1/2} of multiple days), preferably between 10⁻² s⁻¹ and 10⁻⁶ s⁻¹, more preferably between 10⁻³ s⁻¹ and 10⁻⁶ s⁻¹, such as between 10⁻⁴ s⁻¹ and 10⁻⁶ s⁻¹.

Preferably, a binding domain or binding unit present in a compound of the invention is preferably such that it will bind to bind to IL-23 (and in particular for the subunit(s) of IL-23 against which they are directed) with an affinity less than 500 nM, preferably less than 200 nM, more preferably less than 10 nM, such as less than 500 pM.

Thus, in a first aspect, the invention relates to a biparatopic protein or polypeptide construct that is specific for (as defined herein by reference to PCT/EP2008/066365) and/or directed against (as defined herein by reference to PCT/EP2008/066365) IL-23, and that at least comprises:
a) at least one first binding domain, binding unit or binding site that can bind to an epitope of IL-23 that comprises either (i) a stretch of amino acid residues on the p19 subunit of IL-23 that at least comprises the amino acid residues S100. P101 and V102 and/or the amino acid residues F90 and/or P94; and may also comprise the amino acid residues S95, L96 and/or L97; and/or (ii) a stretch of amino acid residues on the p19 subunit of IL-23 that at least comprises the amino acid residues P136, L140, 8143 and L147; and that may also comprise the amino acid residues S134 and/or W137; and/or (iii) a stretch of amino acid residues on the p19 subunit of IL-23 that at least comprises the amino acid residues S27 and H29, and that may also comprise the amino acid residues P30, V32, H34 and/or M35; and preferably any two of (i), (ii) and/or (iii) or all of (i), (ii) and/or (iii);
and
b) at least one second binding domain, binding unit or binding site that can bind to an epitope of IL-23 that comprises a stretch of amino acids on the p19 subunit of IL-23 that at least comprises the amino acid residues L85, G86, S87 and/or the amino acid residues F90 and T91 and/or the amino acid residues S95, L96, L97 and P98 and/or the amino acid residues V102, G103, Q104, H106, A107 and/or L110.

These constructs may optionally further contain one or more suitable linkers, spacers, and/or other amino acid sequences, moieties, residues, binding domains, binding units or binding sites, as for example described in PCT/EP2008/066365.

When reference is made herein to a stretch of amino acid residues that comprises certain amino acid residues on a subunit of IL-23, said stretch of amino acids encompasses said amino acid residues and optionally also at least 7, such as at least 5 of the amino acid residues on either side and directly adjacent to the mentioned amino acid residues. It will also be clear to the skilled person that, where certain amino acid residues are mentioned herein as being very important for the interaction of a subunit of IL-23 with a binding domain, binding unit or binding site that is part of a compound of the invention and are in close proximity to each other in the primary structure of the subunit (for example, F90, P94 and S100, P101, V102; or L85, G86, S87 and F90, T91 and S95, L96, L97, P98 and V102, G103, Q104, H106, A107, L110) that these amino acid residues may form part of a single antigenic determinant, epitope or binding pocket on IL-23 recognized by the binding domain, binding unit or binding site that is part of a compound of the invention. Similarly, it will also be clear to the skilled person that the various amino acid residues are mentioned herein as being very important for the interaction of a subunit of IL-23 with a binding domain, binding unit or binding site that is part of a compound of the invention, even when they are not in close proximity to each other in the primary structure, may still in the tertiary structure of the (folded) subunit form part of the same single antigenic determinant, epitope or binding pocket on IL-23 that is recognized by the binding domain, binding unit or binding site that is part of a compound of the invention.

The first and second binding domain, binding unit or binding site, respectively, present in the compounds of the invention may comprise any binding domain, binding unit or binding site that are capable of binding to the mentioned antigenic determinant or epitope. For example, as mentioned, they may be “amino acid sequences of the invention” according to PCT/EP2008/066365 that are capable of binding to the mentioned antigenic determinant or epitope.

In one aspect, the first binding domain, binding unit or binding site is preferably a binding domain, binding unit or binding site (and in particular, an “amino acid sequence of the invention” according to PCT/EP2008/066365) that can compete with the Nanobody 119A3 (SEQ ID NO: 1898 in PCT/EP2008/066365) for binding to the epitope defined under a) above and/or that can cross-block (as defined in PCT/EP2008/066365) the binding of 119A3 to the epitope defined under a) above.

In one particular aspect, the first binding domain, binding unit or binding site may be an amino acid sequence that comprises any one, two, three or all of the following amino acid residues (and may further be as described herein):
an amino acid residue that can interact with S27, S100, S101 and/or V102 of p19, such as an A, G, S or T residue, and in particular an A residue; and/or
an amino acid residue that can interact with H29, F90, L140, R413 and/or L147 of p19, such as an 5, T or A residue, and in particular an S residue; and/or
an amino acid residue that can interact with P94, L140 and/or 8143 of p19, such as a G or A residue, and in particular a G residue; and/or
an amino acid residue that can interact with P94, P136 and/or L140 of p19, such as an F, Y or W residue, and in particular an F residue.

In a more particular aspect, the first binding domain, binding unit or binding site may be a variant or analog of 119A3, such as, for example and without limitation, a variant or analog that has been obtained through affinity maturation of 119A3; and/or a variant or analog of 119A3 that (essentially) shares at least CDR1 (or at least those residues of CDR1 that are important for the interaction of 119A3 with p19—see Table 1 below) with 119A3 and preferably also (essentially) shares at least CDR3 (or at least those residues of CDR3 that are important for the interaction of 119A3 with p19—see Table 1 below) with 119A3, but that may for example, compared to 119A3 contain one or more substitutions, insertions or deletions (wherein one or more substitutions, insertions or deletions is defined as at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 substitutions, insertions or deletions), in one or more of the framework regions (for example humanizing substitutions as described in PCT/EP2008/066365); or any suitable combination of the foregoing. Such a variant or analog preferably (i) retains at least 80%, more preferably at least 90%, such as at least 95% sequence identity (as defined in PCT/EP2008/066365) with the amino acid sequence of 119A3; and/or (ii) still retains the ability to specifically bind (as defined in PCT/EP2008/066365) to the epitope defined under a); and/or (iii) retains the ability to compete with 119A3 for binding to the epitope defined under a) above and/or to cross-block (as defined in PCT/EP2008/066365) the binding of 119A3 to the epitope defined under a) above.

For example, such a variant of 119A3 may for example, and without limitation, comprise one or more (further) “humanizing” substitutions (as defined herein) and/or comprise one or more of the following substitutions, compared to the sequence of 119A3:
(a) one or more conservative amino acid substitutions; and/or
(b) one or more substitutions in which a “camelid” amino acid residue at a certain position is replaced by a different “camelid” amino acid residue that occurs at said position (for which reference is for example made to Tables A-6 to A-9 from PCT/EP2008/066365, which mention the various Camelid residues that occur as each amino acid position in wild-type VHH's). Such substitutions may even comprise suitable substitutions of an amino acid residue that occurs at a Hallmark position with another amino acid residue that occurring at a Hallmark position in a wild-type VHH (for which reference is for example made to Tables A-6 to A-9 from PCT/EP2008/066365); and/or
(c) one or more substitutions that improve the (other) properties of the protein, such as substitutions that improve the long-term stability and/or properties under storage of the protein. These may for example and without limitation be substitutions that prevent or reduce oxidation events (for example, of methionine residues); that prevent or reduce pyroglutamate formation; and/or that prevent or reduce isomerisation or deamidation of aspartic acids or asparagines (for example, of DG, DS, NG or NS motifs). For such substitutions, reference is for example made to the International application WO 09/095,235, which is generally directed to methods for stabilizing single immunoglobulin variable domains by means of such substitutions, and also gives some specific example of suitable substitutions (see for example pages 4 and 5 and pages 10 to 15). One example of such substitution may be to replace an NS motif at positions 82a and 82b with an NN motif.

or any suitable combination of two or more of any of the foregoing substitutions (a) to (c).

For the purposes described herein, a humanizing substitution can generally be defined as a substitution whereby an amino acid residue that occurs in a framework regions of a camelid V_{HH} domain is replaced by a different amino acid that occurs at the same position in the framework region of a human V_{H} domain (and preferably, a human V_{H}3 domain). Thus, suitable humanizing substitutions will be clear to the skilled person based on the disclosure herein, the disclosure in WO 09/068,627, and from a comparison of the amino acid sequence of a given V_{HH} sequence and one or more human V_{H} sequences.

Reference is for example made to the Tables A-6 to A-9 of WO 09/068,627, which list some of the amino acid residues that have been found to occur in the framework regions of camelid VHH domains, and the corresponding amino acid residue(s) that most often occur in the framework regions of a human V_{H}3 sequence (such as for example, the germline sequences DP-47, DP-51 or DP-29). The humanizing substitutions that can be taken from these Tables are also some of the preferred humanizing substitutions used in the invention; however, it may also be possible to use humanizing substitutions that have been obtained by comparison with other germline sequences (from the V_{H}3 class or sometimes also from other V_{H} classes). As generally known from WO 09/068,627 (and from the patent applications from Applicant and the further prior art mentioned in WO 09/068,627), based on such sequence comparison, particularly suited and/or optimal humanizing substitutions (and combinations thereof) may generally be determined by limited trial and error, i.e. by introducing one or more envisaged humanizing substitutions and testing the humanized variants thus obtained for one or more desired properties, such as melting temperature, affinity, potency, properties upon formatting, expression levels in a desired host organism, and/or other desired properties for VHH domains or Nanobodies or proteins/polypeptides comprising the same, for which again reference is made to WO 09/068,627 and the further patent applications by applicant mentioned therein). For the purposes mentioned herein, it is not excluded that a humanizing substitution may also be introduced at a Camelid Hallmark residue, as long as this essentially does not detract (or does not detract too much) from the desired properties of the variant (in particular, the desired properties of VHH's and Nanobodies, as described in WO 09/068,627). Preferably, however, the humanizing substitutions are not at Camelid Hallmark residues (however, as described in the U.S. provisional application U.S. 61/329,908 by Ablynx N.V specifically for variants of 119A3, variants of 119A3 suitable for use herein may contain one or more of the substitutions H37Y, Q44G, K84R and/or Q108L).

Some particularly suitable variants of 119A3 that may be present in the amino acid sequences of the invention may for example be as described in the U.S. provisional application U.S. 61/329,908 by Ablynx N.V. filed on Apr. 30, 2010 and entitled “Amino acid sequence directed against the p19 subunit of the heterodimeric cytokine IL-23”. As mentioned therein, such variants of 119A3 may:
be a variant of PMP119A3 (SEQ ID NO:1 in U.S. 61/329,908) that comprises, compared to the amino acid sequence of PMP119A3, (i) at least one and preferably both of the mutations H37Y and M43K; (ii) a valine residue at position 78; (iii) at least one, preferably at least two, and more preferably three, four of five humanizing substitutions; (iv) as well as optionally one or more further suitable amino acid substitutions; and may in particular be
a variant of 119A3(H37Y-M43K) (SEQ ID NO:2 in U.S. 61/329,908) that comprises, compared to the amino acid sequence of PMP119A3(H37Y-M43K), (i) a valine residue at position 78; (ii) at least one, preferably at least two, and more preferably three, four of five humanizing substitutions; (iii) as well as optionally one or more further suitable amino acid substitutions; such as for example:
a variant of 119A3v17 (SEQ ID NO:3 in U.S. 61/329,908) that comprises, compared to the amino acid sequence of 119A3v17, (i) a valine residue at position 78; (ii) optionally 1 to 5, such as one, two or three further amino acid differences compared to the sequence of 119A3v17.

Some specific, but non-limiting examples of variants of 119A3 that could be present in the constructs of the present invention as the “first binding domain” are the variants of 119A3 cited in WO 09/068,627, such as P23IL119A3(H137Y) (SEQ ID NO: 2559 in WO 09/068,627), P23IL119A3(M43K) (SEQ ID NO: 2560 in WO 09/068,627), P23IL119A3(H37Y-M43K) (SEQ ID NO: 2560 in WO 09/068,627) and a series of humanized variants of 119A3 (with the H37Y and M43K mutations) called P23IL 119A3-BASIC and P23IL119A3V1 to P23IL119A3V17 (SEQ ID NOs: 2561 to 2579 in WO 09/068,627; as well as the variants of 119A3 described in U.S. provisional application U.S. 61/329,908 by Ablynx N.V., or one of the preferred variants cited in the next paragraph.

In a preferred, but non-limiting aspect of the invention, the “first binding domain” may be chosen from the following variants of 119A3: 119A3v18 (SEQ ID NO:6 in the attached sequence listing), 119A3v20 (SEQ ID NO:7 in the attached sequence listing), 119A3v21 (SEQ ID NO:8 in the attached sequence listing) or 119A3v22 (SEQ ID NO's: 7 in the attached sequence listing).

The second binding domain, binding unit or binding site is preferably a binding domain, binding unit or binding site (and in particular, an “amino acid sequence of the invention” according to PCT/EP2008/066365) that can compete with the Nanobody 81A12 (SEQ ID NO: 1936 in PCT/EP2008/066365) for binding to the epitope defined under b) above and/or that can cross-block (as defined in PCT/EP2008/066365) the binding of 81A1.2 to the epitope defined under b) above.

In a particular aspect, the second binding domain, binding unit or binding site may be an amino acid sequence that comprises any one, two, three, four or all of the following amino acid residues (and may further be as described herein):
an amino acid residue that can interact with P98, G103, Q104 and/or A107 of p19, such as an A or S residue, and in particular an A residue; and/or
an amino acid residue that can interact with G103, H106, A107 and/or L110 of p19, such as a Y, F or W, and in particular a Y residue; and/or
an amino acid residue that can interact with S95, L96, L97 and/or P98 of p19, such as a Y, F or W residue, and in particular a Y residue; and/or
an amino acid residue that can interact with L85, G86, F90, T91, L96, L97, P98, V102, G103 and/or H106 of p19, such as an Y, F or V residue, and in particular a Y residue; and/or
an amino acid residue that can interact with G86, S87 and/or H106 of p19, such as an S, A or T residue, and in particular an S residue.

In a more particular aspect, the second binding domain, binding unit or binding site may be a variant or analog of 81A12, such as, for example and without limitation, a variant or analog that has been obtained through affinity maturation of 81A12; and/or a variant or analog of 81A12 that (essentially) shares at least CDR2 (or at least those residues of CDR2 that are important for the interaction of 81A12 with p19—see Table 2 below) with 81A12 and preferably also (essentially) shares at least CDR3 (or at least those residues of CDR3 that are important for the interaction of 81A12 with p19—see Table 2 below) with 81A12, but that may for example, compared to 81A12 contain one or more substitutions, insertions or deletions in one or more of the framework regions (for example humanizing substitutions as described in PCT/EP2008/066365); or any suitable combination of the foregoing. Such a variant or analog preferably (i) retains at least 80%, more preferably at least 90%, such as at least 95% sequence identity (as defined in PCT/EP2008/066365) with the amino acid sequence of 81A12; and/or (ii) still retains the ability to specifically bind (as defined in PCT/EP2008/066365) to the epitope defined under b); and/or (iii) retains the ability to compete with 81A12 for binding to the epitope defined under b) above and/or to cross-block (as defined in PCT/EP2008/066365) the binding of 81A12 to the epitope defined under b) above.

For example, such a variant of 81A12 may for example, and without limitation, comprise one or more (further) “humanizing” substitutions (as defined herein) and/or comprise one or more of the following substitutions, compared to the sequence of 81A12:
(a) one or more conservative amino acid substitutions; and/or
(b) one or more substitutions in which a “camelid” amino acid residue at a certain position is replaced by a different “camelid” amino acid residue that occurs at said position (for which reference is for example made to Tables A-6 to A-9 from PCT/EP2008/066365, which mention the various Camelid residues that occur as each amino acid position in wild-type VHH's). Such substitutions may even comprise suitable substitutions of an amino acid residue that occurs at a Hallmark position with another amino acid residue that occurding at a Hallmark position in a wild-type VHH (for which reference is for example made to Tables A-6 to A-9 from PCT/EP2008/066365); and/or
(c) one or more substitutions that improve the (other) properties of the protein, such as substitutions that improve the long-term stability and/or properties under storage of the protein. These may for example and without limitation be substitutions that prevent or reduce oxidation events (for example, of methionine residues); that prevent or reduce pyroglutamate formation; and/or that prevent or reduce isomerisation or deamidation of aspartic acids or asparagines (for example, of DG, DS, NG or NS motifs). For such substitutions, reference is for example made to the International application WO 09/095,235, which is generally directed to methods for stabilizing single immunoglobulin variable domains by means of such substitutions, and also gives some specific example of suitable substitutions (see for example pages 4 and 5 and pages 10 to 15). One example of such substitution may be to replace an NS motif at positions 82a and 82b with an NN motif.

or any suitable combination of two or more of any of the foregoing substitutions (a) to (c) (in which such humanizing substitutions can generally be as described herein for the humanizing substitutions that can be present in the variants of 119A3).

Some specific, but non-limiting examples of variants of 81A12 that could be present in the constructs of the present invention as the “second binding domain” are the variants of 81A12 cited in WO 09/068,627, such as P23IL81A12BASIC (SEQ ID NO: 2580 in WO 09/068,627) or one of P23IL81A12v1 to P23IL81A12v5 (SEQ ID NOs: 2581 to 2585 in WO 09/068,627), or 81A12v7 (SEQ ID NO:11); of which 81A12v5 and 81A12v7 are particularly preferred.

In one aspect, a compound of the invention comprises at least one binding domain which is the Nanobody 119A3 (or a variant or analog thereof as defined herein) and at least one binding domain which is the Nanobody 81A12 (or a variant or analog thereof as defined herein). In this aspect, the compound of the invention is not one of the amino acid sequences of SEQ ID NO: 2157, 2543, 2544, 2546, 2547, 2615, 2616, 2617, 2618 or 2622 of PCT/EP2008/066365, but may for example be a construct in which 119A3 (or a variant or analog thereof as defined herein) and 81A12 (or a variant or analog thereof as defined herein) are formatted in another way than in the aforementioned constructs of PCT/EP2008/066365.

For example, in one specific, but non-limiting aspect, a biparatopic protein or polypeptide of the present invention may comprise one binding domain that is a variant or analog of 119A3 (and in particular a humanized variant 119A3, which may for example be as further described herein) and one binding domain which is variant or analog of 81A12 (and in particular a humanized variant 81A12, which may for example be as further described herein), in which the binding domain that is a variant or analog of 81A12 (and in particular a humanized variant 81A12) is towards the N-terminus (i.e. “upstream of”) of the protein or polypeptide compared to the binding domain that is a variant or analog of 119A3 (and in particular a humanized variant 119A3, which may for example be as further described herein). Such biparatopic constructs with the 81A12-based binding unit towards the N-terminus may further essentially be as described in PCT/EP2008/066365; and may for example contain one or more further Nanobodies or other binding units, as well as suitable linkers and other functional groups, all as described in WO 09/068,627. For example, such constructs may be provided with increased half-life, for example through suitable modification such as through pegylation, by fusion to albumin, by including a Nanobody that can bind to serum albumin (such as the Nanobodies Alb-1 or Alb-8 described in WO 09/068,627, or one of the other serum-albumin binding Nanobodies described in WO 08/028,977), or by attachment of a serum albumin binding peptide, such as those described in WO 08/068,280, WO 09/127,691 or further improved variants of such peptides).

Some non-limiting examples of such proteins and polypeptides with the 81A12-based binding unit towards the N-terminus may be represented as follows (with the N-terminus of the polypeptide towards the right and the C-terminus towards the left):
[81A12-based binding domain]-linker-[119A3-based binding domain], which construct may optionally be pegylated for increased half-life;
[81A12-based binding domain]-linker-[Nanobody binding to serum albumin, such as Alb-1 or Alb-8]-linker-[119A3-based binding domain];
[serum albumin]-linker-[81A12-based binding domain]-linker-[119A3-based binding domain];
[8A12-based binding domain]-linker-[119A3-based binding domain]-linker-[serum albumin]
[serum albumin binding peptide (monovalent or in tandem)]-[81A12-based binding domain]-linker-[119A3-based binding domain];
[81A12-based binding domain]-linker-[119A3-based binding domain]-[serum albumin binding peptide (monovalent or in tandem)].

It may be that such constructs in which the 81A12-based binding domain is located towards the N-terminus (i.e. relative to the 119A3-based binding domain) may have one or more favourable properties compared to the corresponding construct in which the 119A3-based binding domain is located towards the N-terminus (i.e. relative to the 81A12-based binding domain). For example, polypeptides in which the 81A12-based binding domain is located towards the N-terminus may show higher expression or production yields compared to corresponding construct in which the 119A3-based binding domain is located towards the N-terminus. Reference is made to Example 5.

Constructs with the 119A3-based binding unit towards the N-terminus may for example be formatted as follows:
[119A3-based binding domain]-linker-[81A12-based binding domain], which construct may optionally be pegylated for increased half-life;
[119A3-based binding domain]-linker-[Nanobody binding to serum albumin, such as Alb-1 or Alb-8]-linker-[81A12-based binding domain];
[serum albumin]-linker-[119A3-based binding domain]-linker-[81A12-based binding domain];
[119A3-based binding domain]-linker-[81A12-based binding domain]-linker-[serum albumin]
[serum albumin binding peptide (monovalent or in tandem)]-[119A3-based binding domain]-linker-[81A12-based binding domain];
[119A3-based binding domain]-linker-[81A12-based binding domain]-[serum albumin binding peptide (monovalent or in tandem)].

Other specific, but non-limiting aspects of the invention are:
constructs as further described herein, that comprise 119A3v18, 119A3v19, 119A3v20 or 119A3v22 as the first binding domain and a variant of 81A12 (as described herein) as the second binding domain. These constructs may for example be formatted and/or half-life extended as indicated in the preceding paragraph.
constructs as further described herein, that comprise a variant of 119A3 (as described herein) as the first binding domain and 81A12v7 as the second binding domain. These constructs may for example be formatted and/or half-life extended as indicated in the preceding paragraph.
constructs as further described herein, that comprise 119A3v18, 119A3v19, 119A3v20 or 119A3 as the first binding domain and 81A12v7 as the second binding domain. These constructs may for example be formatted and/or half-life extended as indicated in the preceding paragraph;
   and again, in all these constructs, it may be preferred to have the 81A12-based binding domain towards the N-terminal end (i.e. upstream of) the 119A3-based binding domain.

Some specific, but non-limiting examples of such constructs are given in SEQ ID NO's: 16 to 27. Based on the disclosure herein, the skilled person will be able to provide other, essentially similar constructs of the invention (for example, with different linkers, different formatting, different half-life extension, or different binding units).

In the above constructs, the first and second binding domain, binding unit and/or binding site may be suitably linked to each other, optionally via one or more suitable linkers (as generally described PCT/EP2008/066365) and/or optionally via one or more other amino acid sequences (which may be other binding domains, binding units or binding sites, for example for increasing the half-life, as further described in PCT/EP2008/066365). Preferably, the first and second binding domain, binding unit and/or binding site are linked to each other in such a way (again, via one or more suitable linkers and/or one or more further amino acid sequences) that the first binding domain, binding unit and/or binding site can bind to the epitope or antigenic determinant referred to under a) above and that the second binding domain, binding unit and/or binding site can bind (i.e. essentially simultaneously) to the epitope or antigenic determinant referred to under b). Also, preferably, the first and second binding domain, binding unit and/or binding site are linked to each other in such a way (again, via one or more suitable linkers and/or one or more further amino acid sequences) that the compounds of the invention preferably undergo intramolecular binding (i.e. with both binding domains, binding units or binding sites binding to the same IL-23 molecule) rather than intermolecular binding (i.e. with both binding domains, binding units or binding sites binding to different IL-23 subunit molecules).

In another aspect, the invention relates to a biparatopic protein or polypeptide construct that is specific for (as defined herein by reference to PCT/EP2008/066365) IL-23 and/or directed against (as defined herein by reference to PCT/EP2008/066365) IL-23, and that comprises at least one binding domain, binding unit or binding site that can bind to the p19/p40 interface of IL-23, and in particular to an epitope of IL-23 that comprises either (i) a stretch of amino acids on the p19 subunit of IL-23 that at least comprises the amino acid residue H163 and optionally also the amino acid residue T167; and/or (ii) stretch of amino acids on the p40 subunit of IL-23 that at least comprises the amino acid residues W240, S241, T242, H244 and/or F247; and/or (iii) a stretch of amino acids on the p40 subunit of IL-23 that at least comprises the amino acid residues N113, Y114, S115 and/or R117; and preferably any two of (i), (ii) and/or (iii) or all of (i), (ii) and/or (iii). In this aspect, the compound of the invention is not one of the amino acid sequences of SEQ ID NO: 1932, 2149, 2159, 2168, 2532, 2534, 2538, 2540, 2545, 2549, 2551, 2552, 2553, 2554, 2556, 2558, 2603-2606 of PCT/EP2008/066365, but may for example be a construct in which 124C4 (or a variant or analog thereof as defined herein) is formatted in another way than in the aforementioned constructs of PCT/EP2008/066365.

In another aspect, the invention relates to a biparatopic protein or polypeptide construct that is specific for (as defined herein by reference to PCT/EP2008/066365) and/or directed against (as defined herein by reference to PCT/EP2008/066365) IL-23, and that at least comprises:
a) at least one first binding domain, binding unit or binding site that can bind to an epitope of IL-23 that comprises either (i) a stretch of amino acid residues on the p19 subunit of IL-23 that at least comprises the amino acid residues K20, T23 and L24 and/or the amino acid residues W26, S27, A28 and H29; and/or (ii) a stretch of amino acid residues on the p19 subunit of IL-23 that at least comprises the amino acid residues E93, P94, S95, L96, L97, P98, D99, S100, P101 and/or V102; and/or (iii) a stretch of amino acid residues on the p19 subunit of IL-23 that at least comprises the amino acid residues W137, L140 and/or 8143; and preferably any two of (i), (ii) and/or (iii) or all of (i), (ii) and/or (iii);

and
b) at least one second binding domain, binding unit or binding site that can bind to the p19/p40 interface of IL-23, and in particular to an epitope of IL-23 that comprises either (i) a stretch of amino acids on the p19 subunit of IL-23 that at least comprises the amino acid residue H163 and optionally also the amino acid residue T167; and/or (ii) stretch of amino acids on the p40 subunit of IL-23 that at least comprises the amino acid residues W240, S241, T242. H244 and/or F247; and/or (iii) a stretch of on the p40 subunit of IL-23 that at least comprises the amino acid residues N113, Y114, S115 and/or R117; and preferably any two of (i), (ii) and/or (iii) or all of (i), (ii) and/or (iii).

These constructs may optionally further contain one or more suitable linkers, spacers, and/or other amino acid sequences, moieties, residues, binding domains, binding units or binding sites, as for example described in PCT/EP2008/066365.

Again, in these compounds of the invention, the first and second binding domain, binding unit or binding site, respectively, present in the compounds of the invention may comprise any binding domain, binding unit or binding site that is capable of binding to the mentioned antigenic determinant or epitope. For example, as mentioned, they may be “amino acid sequences of the invention” according to PCT/EP2008/066365 that are capable of binding to the mentioned antigenic determinant or epitope.

In one particular aspect, the first binding domain, binding unit or binding site may be an amino acid sequence that comprises any one, two, three or all of the following amino acid residues (and may further be as described herein):
an amino acid residue that can interact with S27, S100, S101 and/or V102 of p19, such as an A, G, S or T residue, and in particular an A residue; and/or
an amino acid residue that can interact with H29, F90, L140, R413 and/or L147 of p19, such as an 5, T or A residue, and in particular an S residue; and/or
an amino acid residue that can interact with P94, L140 and/or R143 of p19, such as a G or A residue, and in particular a G residue; and/or
an amino acid residue that can interact with P94, P136 and/or L140 of p19, such as an F, Y or W residue, and in particular an F residue.

In one aspect, the first binding domain, binding unit or binding site is preferably a binding domain, binding unit or binding site (and in particular, an “amino acid sequence of the invention” according to PCT/EP2008/066365) that can compete with the Nanobody 37D5 (SEQ ID NO: 2490 in PCT/EP2008/066365) for binding to the epitope defined under c) above and/or that can cross-block (as defined in PCT/EP2008/066365) the binding of 37D5 to the epitope defined under c) above.

In a more particular aspect, the first binding domain, binding unit or binding site may be a variant or analog of 37D5, such as, for example and without limitation, a variant or analog that has been obtained through affinity maturation of 37D5; and/or a variant or analog of 37D5 that (essentially) shares at least CDR1 (or at least those residues of CDR1 that are important for the interaction of 37D5 with p19—see Table 3 below) with 37D5 and/or (essentially) shares at least CDR2 (or at least those residues of CDR2 that are important for the interaction of 37D5 with p19—see Table 3 below); and/or (essentially) shares at least CDR3 (or at least those residues of CDR3 that are important for the interaction of 37D5 with p19—see Table 3 below) with 37D5, but that may for example, compared to 37D5 contain one or more substitutions, insertions or deletions in one or more of the framework regions (for example humanizing substitutions as described in PCT/EP2008/066365); or any suitable combination of the foregoing. Such a variant or analog preferably (i) retains at least 80%, more preferably at least 90%, such as at least 95% sequence identity (as defined in PCT/EP2008/066365) with the amino acid sequence of 37D5; and/or (ii) still retains the ability to specifically bind (as defined in PCT/EP2008/066365) to the epitope defined under c) above; and/or (iii) retains the ability to compete with 37D5 for binding to the epitope defined under c) above and/or to cross-block (as defined in PCT/EP2008/066365) the binding of 37D5 to the epitope defined under c) above.

For example, such a variant of 37D5 may for example, and without limitation, comprise one or more (further) “humanizing” substitutions (as defined in herein) and/or comprise one or more of the following substitutions, compared to the sequence of 37D5:
(a) one or more conservative amino acid substitutions; and/or
(b) one or more substitutions in which a “camelid” amino acid residue at a certain position is replaced by a different “camelid” amino acid residue that occurs at said position (for which reference is for example made to Tables A-6 to A-9 from PCT/EP2008/066365, which mention the various Camelid residues that occur as each amino acid position in wild-type VHH's). Such substitutions may even comprise suitable substitutions of an amino acid residue that occurs at a Hallmark position with another amino acid residue that occurding at a Hallmark position in a wild-type VHH (for which reference is for example made to Tables A-6 to A-9 from PCT/EP2008/066365); and/or
(c) one or more substitutions that improve the (other) properties of the protein, such as substitutions that improve the long-term stability and/or properties under storage of the protein. These may for example and without limitation be substitutions that prevent or reduce oxidation events (for example, of methionine residues); that prevent or reduce pyroglutamate formation; and/or that prevent or reduce isomerisation or deamidation of aspartic acids or asparagines (for example, of DG, DS, NO or NS motifs). For such substitutions, reference is for example made to the International application WO 09/095,235, which is generally directed to methods for stabilizing single immunoglobulin variable domains by means of such substitutions, and also gives some specific example of suitable substitutions (see for example pages 4 and 5 and pages 10 to 15). One example of such substitution may be to replace an NS motif at positions 82a and 82b with an NN motif.

or any suitable combination of two or more of any of the foregoing substitutions (a) to (c) (in which such humanizing substitutions can generally be as described herein for the humanizing substitutions that can be present in the variants of 119A3).

Some specific, but non-limiting examples of variants of 37D5 that could be present in the constructs of the present invention as the “first binding domain” are the variants of 37D5 cited in WO 09/068,627, such as P23IL37D5V1, P23IL37D5V3 P23IL37D5V6 P23IL37D5V16 or P23IL37D5V17 (see SEQ ID NO's: 2598-2602 in WO 09/068,627) or 37D5v18 (SEQ ID NO:15), of which P23IL37D5V17 and 37D5v18 are particularly preferred.

The second binding domain, binding unit or binding site is preferably a binding domain, binding unit or binding site (and in particular, an “amino acid sequence of the invention” according to PCT/EP2008/066365) that can compete with the Nanobody 124C4 SEQ ID NO: 1932 in PCT/EP2008/066365) for binding to the epitope defined under d) above and/or that can cross-block (as defined in PCT/EP2008/066365) the binding of 124C4 to the epitope defined under d) above.

In one particular aspect, the second binding domain, binding unit or binding site may be an amino acid sequence that comprises any one, two, three, four or all of the following amino acid residues (and may further be as described herein):
an amino acid residue that can interact with P60, R64 and/or H163 of p19 and/or with P243 on p40, such as a D, E, Q or N residue, and in particular a D residue; and/or
an amino acid residue that can interact with T242, P243 and/or H244 of p40, such as a D, E, Q or N residue, and in particular a D residue; and/or
an amino acid residue that can interact with N113, Y114, S115 and/or P178 and/or S241, T242, F247 of p40, such as a G or A, and in particular an A residue; and/or
an amino acid residue that can interact with S115, R117 and/or Q172 and/or W240, S241, T242 of p40, such as a W, F or Y residue, and in particular a W residue.

In a more particular aspect, the second binding domain, binding unit or binding site may be a variant or analog of 124C4, such as, for example and without limitation, a variant or analog that has been obtained through affinity maturation of 124C4; and/or a variant or analog of 124C4 that (essentially) shares at least CDR1 (or at least those residues of CDR1 that are important for the interaction of 124C4 with IL-23—see Table 4 below) with 124C4 and/or (essentially) shares at least CDR2 (or at least those residues of CDR2 that are important for the interaction of 124C4 with IL-23—see Table 4 below); and/or (essentially) shares at least CDR3 (or at least those residues of CDR3 that are important for the interaction of 124C4 with IL-23—see Table 4 below) with 124C4, but that may for example, compared to 124C4 contain one or more substitutions, insertions or deletions in one or more of the framework regions (for example humanizing substitutions as described in PCT/EP2008/066365); or any suitable combination of the foregoing. Such a variant or analog preferably (i) retains at least 80%, more preferably at least 90%, such as at least 95% sequence identity (as defined in PCT/EP2008/066365) with the amino acid sequence of 124C4; and/or (ii) still retains the ability to specifically bind (as defined in PCT/EP2008/066365) to the epitope defined under d) above; and/or (iii) retains the ability to compete with 124C4 for binding to the epitope defined under d) above and/or to cross-block (as defined in PCT/EP2008/066365) the binding of 124C4 to the epitope defined under d) above.

For example, such a variant of 124C4 may for example, and without limitation, comprise one or more (further) “humanizing” substitutions (as defined in herein) and/or comprise one or more of the following substitutions, compared to the sequence of 124C4:
(a) one or more conservative amino acid substitutions; and/or
(b) one or more substitutions in which a “camelid” amino acid residue at a certain position is replaced by a different “camelid” amino acid residue that occurs at said position (for which reference is for example made to Tables A-6 to A-9 from PCT/EP2008/066365, which mention the various Camelid residues that occur as each amino acid position in wild-type VHH's). Such substitutions may even comprise suitable substitutions of an amino acid residue that occurs at a Hallmark position with another amino acid residue that occurding at a Hallmark position in a wild-type VHH (for which reference is for example made to Tables A-6 to A-9 from PCT/EP2008/066365); and/or
(c) one or more substitutions that improve the (other) properties of the protein, such as substitutions that improve the long-term stability and/or properties under storage of the protein. These may for example and without limitation be substitutions that prevent or reduce oxidation events (for example, of methionine residues); that prevent or reduce pyroglutamate formation; and/or that prevent or reduce isomerisation or deamidation of aspartic acids or asparagines (for example, of DG, DS, NG or NS motifs). For such substitutions, reference is for example made to the International application WO 09/095,235, which is generally directed to methods for stabilizing single immunoglobulin variable domains by means of such substitutions, and also gives some specific example of suitable substitutions (see for example pages 4 and 5 and pages 10 to 15). One example of such substitution may be to replace an NS motif at positions 82a and 82b with an NN motif.

or any suitable combination of two or more of any of the foregoing substitutions (a) to (c) (in which such humanizing substitutions can generally be as described herein for the humanizing substitutions that can be present in the variants of 119A3).

Some specific, but non-limiting examples of variants of 124C4 that could be present in the constructs of the present invention as the “first binding domain” are the variants of 124C4 cited in WO 09/068,627, such as P23IL124C4-BASIC, P23IL124C4V1 P23IL124C4V2 or P23IL124C4V3 (see SEQ ID NO's: 2603-2605 in WO 09/068,627) or one of 124C4v5 (SEQ ID NO:12), 124C4v6 (SEQ ID NO:13) or 124C4v7 (SEQ ID NO: 14); of which 124C4v5, 124C4v6 and 24C4v7 are particularly preferred. [In this respect, it should be noted that the molecule referred to as 124C4v7 herein (SEQ ID NO: 14) has an amino acid sequence that is different from the molecule called “P23IL124C4V7” (SEQ ID NO: 2614) in PCT/EP2008/066365 (which is a humanized variant of the molecule called “P23IL20B11” (SEQ ID NO:2502) described in PCT/EP2008/066365. When reference is made herein to “124C4v7”, what is meant is the sequence from SEQ ID NO: 7, not the sequence of SEQ ID NO: 2614 from PCT/EP2008/066365].

In one aspect, a compound of the invention comprises at least one binding domain which is the Nanobody 37D5 (or a variant or analog thereof as defined herein) and at least one binding domain which is the Nanobody 124C4 (or a variant or analog thereof as defined herein). In this aspect, the compound of the invention is not one of the amino acid sequences of SEQ ID NO: 2549, 2551, 2552, 2556 or 2558 of PCT/EP2008/066365, but may for example be a construct in which 37D5 (or a variant or analog thereof as defined herein) and 124C4 (or a variant or analog thereof as defined herein) are formatted in another way than in the aforementioned constructs of PCT/EP2008/066365.

For example, in one specific, but non-limiting aspect, a biparatopic protein or polypeptide of the present invention may comprise one binding domain that is a variant or analog of 37D5 (and in particular a humanized variant 37D5, which may for example be as further described herein) and one binding domain which is variant or analog of 124C4 (and in particular a humanized variant 124C4, which may for example be as further described herein), in which the binding domain that is a variant or analog of 124C4 (and in particular a humanized variant 124C4) is towards the N-terminus (i.e. “upstream of”) of the protein or polypeptide compared to the binding domain that is a variant or analog of 37D5 (and in particular a humanized variant 37D5, which may for example be as further described herein). Such biparatopic constructs with the 124C4-based binding unit towards the N-terminus may further essentially be as described in PCT/EP2008/066365; and may for example contain one or more further Nanobodies or other binding units, as well as suitable linkers and other functional groups, all as described in WO 09/068,627. For example, such constructs may be provided with increased half-life, for example through suitable modification such as through pegylation, by fusion to albumin, by including a Nanobody that can bind to serum albumin (such as the Nanobodies Alb-1 or Alb-8 described in WO 09/068,627, or one of the other serum-albumin binding Nanobodies described in WO 08/028,977), or by attachment of a serum albumin binding peptide, such as those described in WO 08/068,280, WO 09/127,691 or further improved variants of such peptides).

The constructs that comprise binding units based on 37D5 and 124C4 may for example be formatted as follows:
[124C4-based binding domain]-linker-[37D5-based binding domain], which construct may optionally be pegylated for increased half-life;
[124C4-based binding domain]-linker-[Nanobody binding to serum albumin, such as Alb-1 or Alb-8]-linker-[37D5-based binding domain];
[serum albumin]-linker-[124C4-based binding domain]-linker-[37D5-based binding domain];
[124C4-based binding domain]-linker-[37D5-based binding domain]-linker-[serum albumin]
[serum albumin binding peptide (monovalent or in tandem)]-[124C4-based binding domain]-linker-[37D5-based binding domain];
[124C4-based binding domain]-linker-[37D5-based binding domain]-[serum albumin binding peptide (monovalent or in tandem)].
[37D5-based binding domain]-linker-[124C4-based binding domain], which construct may optionally be pegylated for increased half-life;
[37D5-based binding domain]-linker-[Nanobody binding to serum albumin, such as Alb-1 or Alb-8]-linker-[124C4-based binding domain];
[serum albumin]-linker-[37D5-based binding domain]-linker-[124C4-based binding domain];
[37D5-based binding domain]-linker-[124C4-based binding domain]-linker-[serum albumin]
[serum albumin binding peptide (monovalent or in tandem)]-[37D5-based binding domain]-linker-[124C4-based binding domain];
[37D5-based binding domain]-linker-[124C4-based binding domain]-[serum albumin binding peptide (monovalent or in tandem)].

Other specific, but non-limiting aspects of the invention are:
constructs as further described herein, that comprise 37D5v18 as the first binding domain and a variant of 124C4 (as described herein) as the second binding domain. These constructs may for example be formatted and/or half-life extended as indicated in the preceding paragraph.
constructs as further described herein, that comprise a variant of 37D5 (as described herein) as the first binding domain and 124C4v5, 124C4v6 or 124C4v7 as the second binding domain. These constructs may for example be formatted and/or half-life extended as indicated in the preceding paragraph.
constructs as further described herein, that comprise 37D5v18 as the first binding domain and 124C4v5, 124C4v6 or 124C4v7 as the second binding domain. These constructs may for example be formatted and/or half-life extended as indicated in the preceding paragraph.

Some specific, but non-limiting examples of such constructs are given in SEQ ID NO's: 28 to 30. Based on the disclosure herein, the skilled person will be able to provide other, essentially similar constructs of the invention (for example, with different linkers, different formatting, different half-life extension, or different binding units).

In the above constructs, the first and second binding domain, binding unit and/or binding site may be suitably linked to each other, optionally via one or more suitable linkers (as generally described PCT/EP2008/066365) and/or optionally via one or more other amino acid sequences (which may be other binding domains, binding units or binding sites, for example for increasing the half-life, as further described in PCT/EP2008/066365). Preferably, the first and second binding domain, binding unit and/or binding site are linked to each other in such a way (again, via one or more suitable linkers and/or one or more further amino acid sequences) that the first binding domain, binding unit and/or binding site can bind to the epitope or antigenic determinant referred to under c) above and that the second binding domain, binding unit and/or binding site can bind (i.e. essentially simultaneously) to the epitope or antigenic determinant referred to under d). Also, preferably, the first and second binding domain, binding unit and/or binding site are linked to each other in such a way (again, via one or more suitable linkers and/or one or more further amino acid sequences) that the compounds of the invention preferably undergo intramolecular binding (i.e. with both binding domains, binding units or binding sites binding to the same IL-23 subunit molecule) rather than intermolecular binding (i.e. with both binding domains, binding units or binding sites binding to different IL-23 subunit molecules).

The compounds of the invention are further preferably such that they can modulate (as defined in PCT/EP2008/066365) the signaling that is mediated by IL-23 and/or its cognate receptor, to modulate (as defined herein) the biological pathways in which IL-23 and/or its cognate receptors are involved, and/or to modulate (as defined herein) the biological mechanisms, responses and effects associated with IL-23, its cognate receptor, such signaling and/or these pathways. Reference is again made to PCT/EP2008/066365. In particular, such modulation may be such that such signaling and/or the biological effects associated with such signaling are reduced (i.e. compared to the signaling or effect without the presence of the compound of the invention, and by at least 1%, such as by at least 5%, for example by at least 10%, at least 30%, at least 50%, at least 70% and up to 90% or more, as determined by a suitable assay, such as one of the assays mentioned in PCT/EP2008/066365)

The amino acid sequences that form the binding domains, binding units or binding sites that are present in the compounds of the invention can be obtained using the techniques that are generally described in PCT/EP2008/066365.

The compounds of the invention may optionally contain one or more further groups, residues, moieties, amino acid sequences, binding domains and/or binding units that confer at least one desired property to the compounds of the invention, such as an increased half-life. Such groups, residues, moieties, amino acid sequences, binding domains and/or binding units may be as further described in PCT/EP2008/066365.

In the compounds of the invention, the at least two binding units that bind to different epitopes or antigenic determinants on IL-23 and the optional further groups, residues, moieties, amino acid sequences, binding domains and/or binding units that make up the compounds of the invention may be suitably linked to each other, for example by direct chemical and/or covalent linkers or via one or more suitable linkers or spacers. Reference is again made to PCT/EP2008/066365. Also, the linkers and the optional further groups, residues, moieties, amino acid sequences, binding domains and/or binding units that make up the compounds of the invention are preferably further such that the final compound of the invention is a fusion protein or polypeptide.

It will be clear to the skilled person from the disclosure herein that the amino acid sequences of the invention are directed against IL-23. Thus, the amino acid sequences of the invention can be used for the same purposes, uses and applications as described in WO 09/068,627, for example to modulate signaling that is mediated by IL-23 and/or its receptor(s); and/or in the prevention or treatment of diseases associated with IL-23 and/or with signaling that is mediated by IL-23, such as for example inflammation and inflammatory disorders such as bowel diseases (colitis, Crohn'disease, IBD), infectious diseases, psoriasis, cancer, autoimmune diseases (such as MS), carcoidis, transplant rejection, cystic fibrosis, asthma, chronic obstructive pulmonary disease, rheumatoid arthritis, viral infection, common variable immunodeficiency, and the various diseases and disorders mentioned in the prior art cited herein. Further reference is made to WO 09/068,627.

The various polypeptides of the invention preferably have a neutralizing activity (expressed as IC50) in a mouse splenocyte assay using hIL-23 (see Example 30 of WO 09/068,627) that is better than (i.e. less than) 50 pM, preferably better than 20 pM, more preferably better than 10 pM such as between 8 and 1 pM or less.

The various constructs of the invention also preferably have a melting point (Tm) determined using DSC (under the conditions set out in Example 5) of more than 60° C.

Possible applications and uses of the amino acid sequences of the invention (and of compositions comprising the same) are mentioned throughout WO 09/068,627 (see for example pages 7/8, 32 and 328 to 337 of WO 09/068,627).

Generally, these may include use in (pharmaceutical composition for) the prevention and/or treatment of diseases and disorders associated with heterodimeric cytokines and their receptors (and in particular, with IL-23 or IL-23 mediated signaling), which as mentioned in WO 09/068,627 are diseases and disorders that can be prevented and/or treated, respectively, by suitably administering to a subject in need thereof (i.e. having the disease or disorder or at least one symptom thereof and/or at risk of attracting or developing the disease or disorder) of either a polypeptide or composition of the invention (and in particular, of a pharmaceutically active amount thereof) and/or of a known active principle active against heterodimeric cytokines (and in particular, IL-23) and/or their receptors or a biological pathway or mechanism in which heterodimeric cytokines (and in particular, IL-23) and/or their receptors is involved (and in particular, of a pharmaceutically active amount thereof). Examples of such diseases and disorders associated with heterodimeric cytokines and their receptors will be clear to the skilled person based on the disclosure herein, and for example include the following diseases and disorders: inflammation and inflammatory disorders such as bowel diseases (colitis, Crohn'disease, IBD), infectious diseases, psoriasis, cancer, autoimmune diseases (such as MS), carcoidis, transplant rejection, cystic fibrosis, asthma, chronic obstructive pulmonary disease, rheumatoid arthritis, viral infection, common variable immunodeficiency, and the various diseases and disorders mentioned in the prior art cited herein. Based thereon, it will also be clear to the skilled person with heterodimeric cytokines (and/or receptors thereof) are involved in which specific diseases and disorders.

For example, as mentioned on pages 4-5 of WO 09/068,627, IL23 was shown to be responsible for the chronic inflammation observed in inflammatory bowel disease. This was confirmed by the fact that the IL23R gene was identified as being involved in inflammatory bowel disease. It has also been found that p19 knock out mice are resistant to collagen-induced arthritis and colitis, whereas comparable p35 knock out mice were found to be more susceptible to collagen-induced arthritis. Also, when p19 knock out mice were crossed with IL-10 knock out mice, the resulting offspring were resistant to colitis, whereas similar crosses of p19 knock out mice with IL-10 knock out mice resulted in offspring that was susceptible to colitis. It was further found that a monoclonal antibody against p19 inhibits the development of EAR, a preclinical animal model for multiple sclerosis, and reduces serum levels of IL-17 (which is not regulated by IL-12). Also, IL-23 rather than IL-12 appears to be the essential cytokine in CNS autoimmune inflammation. All this results suggests that IL-23/p19 may be an attractive target for the treatment of colitis, Crohn's diseases, IBD, multiple sclerosis, rheumatoid arthritis and some of the other diseases and disorders mentioned herein. Also, IL23 and IL27—two of the other heterodimeric cytokines from the IL-12 family—also regulate TH1-cell response, albeit with distinct functions. The ability of IL-23 to stimulate CD4+ T cells to produce IL-17 also has been described as having a dominant role in the development and maintenance of autoimmune inflammation.

Also, Example 45 of WO 09/068,627 shows that the polypeptides of WO 09/068,627 (and thus, by extension, the polypeptides of the invention) can also be valuable in the prevention and treatment of psoriasis (either by systemic/parenteral administration or by topical treatment, e.g. using a crème or lotion (see page 328 and 331-332 of WO 09/068,627).

The invention further relates to nucleic acids encoding the compounds of the invention (i.e. when the compounds of the invention are in the form of a fusion protein or polypeptide); to methods for preparing the compounds of the invention; to host cells expressing or capable of expressing the compounds of the invention; to compositions, and in particular to pharmaceutical compositions, that comprise the compounds of the invention; and to uses of the compounds of the invention and the aforementioned nucleic acids, host cells and/or compositions, in particular for prophylactic, therapeutic or diagnostic purposes, such as the prophylactic, therapeutic or diagnostic purposes mentioned herein. All these may be essentially as generally described in PCT/EP2008/066365 for the biparatopic constructs described therein (but comprising, encoding, expressing, providing or employing a compound of the invention as described herein).

In the Examples below, each crystal structure was determined as follows: the purified proteins were used in crystallization trials employing both a standard screen of approximately 1200 different conditions, as well as crystallization conditions identified using literature data. Conditions initially obtained have been optimized using standard strategies, systematically varying parameters critically influencing crystallization, such as temperature, protein concentration, drop ratio, etc. These conditions were also refined by systematically varying pH or precipitant concentrations. Crystals were obtained via the method of co-crystallization.

Crystals have been flash-frozen and measured at a temperature of 100K. The X-ray diffraction have been collected at the SWISS LIGHT SOURCE (SLS, Villigen, Switzerland) using cryogenic conditions. Data were processed using the programs XDS and XSCALE. The phase information necessary to determine and analyze the structure was obtained by molecular replacement. Subsequent model building and refinement was performed with the software packages CCP4 and COOT. The peptide parameterization was carried out with the program CHEMSKETCH.

Modeling of the interaction was performed using ICM-Pro (Molsoft) and Discovery Studio (Accelrys) with a force-field that is based on the parameters as described in Momany et al. (Momany et al. J. Phys. Chem. 1975, 79, 2361-2381).

For the sequence of the p19 and the p40 subunit of IL-23, reference is made to the following Genbank entries:

for p19: NM_016584 (DNA) -> NP_057668 (protein):
(SEQ ID NO: 1)
mlgsravmlllllpwtaqgravpggsspawtqcqqlsqklctlawsahplvghmdlreegdeettndvphiqcgdgc
dpqglrdnsqfclqrihqglifyekllgsdiftgepsllpdspvgqlhasllglsqllqpeghhwetqqipslspsqpwqrll
lrfkilrslqafvavaarvfahgaatlsp
for p40: NM_002187 (DNA) -> NP_002178 (protein):
(SEQ ID NO: 2)
mchqqlviswfslvflasplvaiwelkkdvyvveldwypdapgemvvltcdtpeedgitwtldqssevlgsgktltiq
vkefgdagqytchkggevlshsllllhkkedgiwstdilkdqkepknktflrceaknysgrftcwwlttistdltfsvkssr
gssdpqgvtcgaatlsaervrgdnkeyeysvecqedsacpaaeeslpievmvdavhklkyenytssffirdiikpdppk
nlqlkplknsrqvevsweypdtwstphsyfsltfcvqvqgkskrekkdrvftdktsatvicrknasisvraqdryysssw
sewasvpcs.

For the p19 sequence, it should be noted that, as mentioned in the relevant Genbank entry, the first 19 amino acid residues in the above sequence form the signal peptide. The mature sequence of p19 comprises amino acid residues 20 to 189 of the sequence given above, and is as follows:

p19 - mature protein:
(SEQ ID NO: 3)
ravpggsspawtqcqqlsqklctlawsahplvghmdlreegdeettndvphiqcgdgcdpqglrdnsqfclqrihqgli
fyekllgsdiftgepsllpdspvgqlhasllglsqllqpeghhwetqqipslspsqpwqrlllrfkilrslqafvavaarvfah
gaatlsp

In the Examples below, the numbering is made with reference to the sequence of SEES ID NO:3 (mature protein without signal peptide).

For the p40 sequence, as also mentioned in the relevant Genbank entry, the first 22 amino acid residues in the above sequence form the signal peptide. The mature sequence of p40 comprises amino acid residues 23 to 328 of the sequence given above, and is as follows:

p40 - mature protein:
(SEQ ID NO: 4)
iwelkkdvyvveldwypdapgemvvltcdtpeedgitwtldqssevlgsgktltiqvkefgdagqytchkggevlshs
llllhkkedgiwstdilkdqkepknktflrceaknysgrftcwwlttistdltfsvkssrgssdpqgvtcgaatlsaervrgd
nkeyeysvecqedsaepaaeeslpievmvdavhklkyenytssffirdiikpdppknlqlkplknsrqvevsweypdt
wstphsyfsltfcvqvqgkskrekkdrvftdktsatvicrknasisvraqdryyssswsewasvpcs

In the Examples below, the numbering is made with reference to the sequence of SEQ ID NO:4 (mature protein without signal peptide).

### EXAMPLE 1

### Binding Interaction Between Nanobody 119A3 and the p19 Subunit

The binding interaction between the Nanobody 119A3 (SEQ ID NO:1898 in PCT/EP2008/066365) and IL-23 was determined by X-ray crystallography and in silico modeling as described herein.

The most relevant binding interactions (based upon the total binding energies) are given in Table 1 below. Of these, the interactions of the residues A31b, S31c, G31d and F31g are the most relevant, as judged by the total binding energy. The further residues indicated in Table 1 are also considered to make a significant contribution to the binding interaction, but less significant than that of the aforementioned residues.

In the sequence below, the (main) amino acid residues in p19 that undergo a binding interaction with 119A3 have been indicated in UPPERCASE (see also Table 1). The most important residues in p19 for the interaction with 119A3 have been indicated in bold.

(SEQ ID NO: 3)
ravpggsspawtqcqqlsqklctlawSaHPlVgHMdlreegdeettndvphiqcgdgcdpqglrdnsqfclqrihq
glifyekllgsdiFtgePSLLpdSPVgqlhasllglsqllqpeghhwetqqipslspSqPWqrLllRfkiLrslqaf
vavaarvfahgaatlsp

Table 1 also lists, for each amino acid residue of 119A3 listed in Table 1, alternative amino acid residues that could, if present on the same position in 119A3, undergo similar interactions with the corresponding amino acid residues in p19 as the amino acid residue that is present at that position in 119A3.

119A3 has no meaningful interactions with the p40 subunit of IL-23.

TABLE 1
Binding interactions of Nanobody 119A3 and the p19 subunit of IL-23

| Amino acid | | | | Other residues that |
|---|---|---|---|---|
| residue in 119A3 | P19 residues | Binding | | potentially can |
| (numbering | interacting with the | energy | Main | undergo similar |
| according to | corresponding | (total in | interaction | interactions with |
| Kabat) | residue in 119A3 | kcal/mol) | type | p19 |
| R27 | V32, M35 | −1.9 | Van der Waals | K |
| I28 | H34, L140 | −1.3 | Van der Waals | V, L, M |
| P31a | H29, P30, L140 | −1.4 | Van der Waals | A, T |
| A31b | S27, S100, P101, | −3.1 | Van der Waals | G, S, T |
| | V102 | | | |
| S31c | H29, F90, L140, | −10.4 | Van der | T, A |
| | R143, L147 | | Waals, H- | |
| | | | bond, | |
| | | | electrostatic | |
| G31d | P94, L140, R143 | −3.4 | Van der Waals | A |
| N31e | L96, S100 | −1.2 | Van der Waals | Q, E |
| I31f | P94, S95, L97 | −0.8 | Van der Waals | V, L, M |
| F31g | P94, P136, L140 | −4.1 | Van der Waals | Y, W |
| L31i | P94, P136 | −1.1 | Van der Waals | V, I, M |
| S97 | S134, P136 | −2.7 | Van der Waals | T, A |
| G98 | P136, W137 | −1.5 | Van der Waals | A |
| S99 | P136, W137 | −1.7 | Van der | A, T |
| | | | Waals, | |
| | | | electrostatic | |

### EXAMPLE 2

### Binding Interaction Between Nanobody 81A12 and the p19 Subunit

The binding interaction between the Nanobody 81A12 (SEQ ID NO:1936 in PCT/EP2008/066365) and IL-23 was determined by X-ray crystallography and in silica modeling as described herein.

The most relevant binding interactions (based upon the total binding energies) are given in Table 2 below. Of these, the interactions of the residues A56, Y58, Y99, Y100 and S100c are the most relevant, as judged by the total binding energy. The residues Q52A, T55, Y59, D61, K64, P98, G100b and Y100e are also considered to make a significant contribution to the binding interaction, but less significant than that of the aforementioned residues. The residues S52, G53 and R100a show some interactions with p19, but less relevant than the aforementioned residues.

in the sequence below, the (main) amino acid residues in p19 that undergo a binding interaction with 81A12 have been indicated in UPPERCASE (see also Table 2). The most important residues in p19 for the interaction with 81A12 have been indicated in bold.

(SEQ ID NO: 3)
ravpggsspawtqcqqlsqklctlawsahplvghmdlreegdeettndvphiqcgdgcdpqglrdnsqfclqrihqgli
fyeklLGSdiFTgepSLLPDspVGQlHAslLglsQllqPEghhwetqqipslspsqpwqrlllrfkilrslq
afvavaarvfahgaatlsp

Table 2 also lists, for each amino acid residue of 81A12 listed in Table 2, alternative amino acid residues that could, if present on the same position in 81A12, undergo similar interactions with the corresponding amino acid residues in p19 as the amino acid residue that is present at that position in 81A12.

81A12 has no meaningful interactions with the p40 subunit of IL-23.

TABLE 2
Binding interactions of Nanobody 81A12 and the p19 subunit of IL-23

| | | | | Other residues |
|---|---|---|---|---|
| Amino acid | | | | that potentially |
| residue in 81A12 | p19 residues | | | can undergo |
| (numbering | interacting with the | Binding | | similar |
| according to | corresponding | energy | Main interaction | interactions with |
| Kabat) | residue in 81A12 | (total) | type | p19 |
| S52 | P98, D99 | −2.0 | Van der Waals, | A, T |
| | | | electrostatic | |
| Q52a | P98, D99 | −4.3 | Van der Waals, | D, E, N |
| | | | electrostatic | |
| G53 | D99 | −2.5 | Van der Waals | A |
| T55 | D99, Q104 | −5.2 | Van der Waals, H- | S |
| | | | bond, electrostatic | |
| A56 | P98, G103, Q104, | −4.2 | Van der Waals | S |
| | A107 | | | |
| Y58 | G103, H106, A107, | −7.7 | Van der Waals, H- | F, W |
| | L110 | | bond, electrostatic | |
| Y59 | Q114 | −2.9 | Van der Waals, H- | F, W |
| | | | bond, electrostatic | |
| D61 | Q114, P118, E119 | −3.9 | Van der Waals, H- | E, Q, N |
| | | | bond, electrostatic | |
| K64 | P118 | −9.2 | Van der Waals | R |
| P98 | L97, P98 | −1.8 | Van der Waals | A |
| Y99 | S95, L96, L97, P98 | −7.3 | Van der Waals | F, W |
| Y100 | L85, G86, F90, T91, | −17.0 | Van der Waals, H- | F, W |
| | L96, L97, P98, V102, | | bond, electrostatic | |
| | G103, H106 | | | |
| R100a | T91, P98 | −1.2 | Van der Waals | K |
| G100b | G86, T91, H106 | −2.3 | Van der Waals, | A |
| | | | electrostatic | |
| S100c | G86, S87, H106 | −6.3 | Van der Waals, H- | A, T |
| | | | bond, electrostatic | |
| Y100e | H106, L110 | −1.7 | Van der Waals | F, W |

### EXAMPLE 3

### Binding Interaction Between Nanobody 37D5 and the p19 and p40 Subunits

The binding interaction between the Nanobody 37D5 (SEQ ID NO:2490 in PCT/EP2008/066365) and IL-23 was determined by X-ray crystallography and in silico modeling as described herein.

The most relevant binding interactions (based upon the total binding energies) are given in Table 3 below. Of these, the interactions of the residues Y31, Y56, P96, E97, C98, Y99, R100b and T101 are the most relevant, as judged by the total binding energy. The residues T28, L32 and S52a, as well as S76 (which binds to R266 in p40) are also considered to make a significant contribution to the binding interaction, but less significant than that of the aforementioned residues. The other residues mentioned in Table 3 show some interactions with p19, but less relevant than the aforementioned residues.

In the sequence below, the (main) amino acid residues in p19 that undergo a binding interaction with 37D5 have been indicated in UPPERCASE (see also Table 3). The most important residues in p19 for the interaction with 37D5 have been indicated in bold.

(SEQ ID NO: 3)
ravpggsspawtqcqqlsqKlcTLaWSAHplvghmdlreegdeettndvphiqcgdgcdpqglrdnsqfclqri
hqglifyekllgsdiftgEPSLLPDSPVgqlhasllglsqllqpeghhwetqqipslspsqpWqrLllRfkilrslq
afvavaarvfahgaatlsp

In the sequence below, the amino acid residue in p40 that undergo a binding interaction with 37D5 have been indicated in UPPERCASE (see also Table 3).

(SEQ ID NO: 4)
iwelkkdvyvveldwypdapgemvvltcdtpeedgitwtldqssevlgsgktltiqvkefgdagqytchkggevlshs
llllhkkedgiwstdilkdqkepknktflrceaknysgrftcwwlttistdltfsvkssrgssdpqgvtcgaatlsaervrgd
nkeyeysvecqedsacpaaeeslpievmvdavhklkyenytssffirdiikpdppknlqlkplknsrqvevsweypdt
wstphsyfsltfcvqvqgkskrekkdRvftdktsatvicrknasisvraqdryyssswsewasvpcs

Table 3 also lists, for each amino acid residue of 37D5 listed in Table 3, alternative amino acid residues that could, if present on the same position in 37D5, undergo similar interactions with the corresponding amino acid residues in p19 or p40, respectively, as the amino acid residue that is present at that position in 37D5.

TABLE 3
Binding interactions of Nanobody 37D5 and the p19 or p40 subunit of IL-23

| Amino acid residue | | | | | | Other residues that |
|---|---|---|---|---|---|---|
| in 37D5 | p19 residues | | p40 residues | | | potentially can |
| (numbering | interacting with | Binding | interacting with | Binding | Main | undergo similar |
| according to | the corresponding residue | energy | the corresponding residue | energy | interaction | interactions with p19 |
| Kabat) | in 37D5 | (total) | in 37D5 | (total) | type | or p40 |
| A1 | | — | Q289, W297 | −1.3 | Van der Waals | |
| V2 | W26, H29 | −1.2 | | — | Van der Waals | |
| S25 | | — | R287 | −1.0 | Van der Waals | A, T |
| G26 | | — | R266, F268, R287 | −3.0 | Van der Waals | A |
| F27 | W26 | −0.8 | F268, Q289, S294, W297 | −2.3 | Van der Waals | Y, W |
| T28 | W26 | −1.7 | | — | Van der Waals | S, A |
| Y31 | K20, T23, L24, W26, S27 | −6.9 | | — | Van der Waals, | F, W |
| | | | | | H-bond, | |
| | | | | | electrostatic | |
| L32 | W26, S27, H29 | −3.5 | | — | Van der Waals | I, V, M |
| S52 | L97, D99 | −2.5 | | — | Van der Waals, | A, T |
| | | | | | H-bond, | |
| | | | | | electrostatic | |
| S52a | D99 | −1.0 | | — | Van der Waals, | A, T |
| | | | | | electrostatic | |
| S53 | D99 | −1.1 | | — | Van der Waals, | T |
| | | | | | electrostatic | |
| Q55 | D99 | −1.4 | | — | Van der Waals, | D, E, N |
| | | | | | electrostatic | |
| Y56 | L97, P98, D99 | −4.3 | | — | Van der Waals | F, W |
| E75 | | — | R266 | −2.9 | Van der Waals | |
| S76 | | — | R266 | −1.5 | Van der Waals, | |
| | | | | | electrostatic | |
| T94 | H29 | −1.4 | | — | Van der Waals | S, A |
| P96 | A28, H29 | −2.0 | | — | Van der Waals | A |
| E97 | L96, L97, S100, P101, V102 | −5.8 | | — | Van der Waals, | D, Q, N |
| | | | | | H-bond, | |
| | | | | | electrostatic | |
| C98 | L97 | −1.0 | | — | Van der Waals | |
| Y99 | P94, S95, L96, L140, R143 | −8.6 | | — | Van der Waals, | F, W |
| | | | | | H-bond, | |
| | | | | | electrostatic | |
| R100b | P94, S95, L97 | −4.8 | | — | Van der Waals, | K |
| | | | | | H-bond, | |
| | | | | | electrostatic | |
| Y101 | E93, P94, W137, L140 | −3.2 | | — | Van der Waals | F, W |

### EXAMPLE 4

### Binding Interaction Between Nanobody 124C4 and the p19 and p40 Subunits

The binding interaction between the Nanobody 124C4 (SEQ ID NO:1932 in PCT/EP2008/066365) and IL-23 was determined by X-ray crystallography and in silico modeling as described herein.

The most relevant binding interactions (based upon the total binding energies) are given in Table 4 below. Of these, the interactions of the residues D30, D51, G98 and G99 are the most relevant, as judged by the total binding energy. The residues T27b, D29, S56, A57, T97, G100, L100a and Y100f are also considered to make a significant contribution to the binding interaction, but less significant than that of the aforementioned residues. The other residues mentioned in Table 4 show some interactions with p19 and/or p40, but less relevant than the aforementioned residues. It can be seen that 124C4 binds to amino acid residues in p19 and p40 that in the heterodimer IL-23 lie at, or close to, the p19/p40 interface.

In the sequence below, the (main) amino acid residues in p19 that undergo a binding interaction with 124C4 have been indicated in UPPERCASE (see also Table 4). The most important residues in p19 for the interaction with 124C4 have been indicated in bold.

(SEQ ID NO: 3)
ravpggsspawtqcqqlsqklctlawsahplvghmdlreegdeettndvphiqcgdgcDPQglRdnsqfclqrihq
glifyekllgsdiftgepsllpdspvgqlhasllglsqpegHhweTqqipslspsqpwqrlllrfkilrslqafvavaar
vfahgaatlsp

In the sequence below, the (main) amino acid residues in p40 that undergo a binding interaction with 124C4 have been indicated in UPPERCASE (see also Table 4). The most important residues in p19 for the interaction with 124C4 have been indicated in bold.

(SEQ ID NO: 4)
iwelkkdvyvveldwypdapgemvvltcdtpeedgitwtldqssevlgsgktltiqvkefgdagqytchkggevlshs
llllhkkedgiwstdilkdqkepknktflrceakNYSgRftcwwlttistdltfsvkssrgssdpqgvtcgaatlsaervr
gdnkeyeysvecQedsacPaaeeslpievmvdavhklkyenytssffirdiikpdppKnlqlkplknsrqvevswE
YpDTWSTPHSyFsltfcvqvqgkskrekkdRvftdktsatvicrknasisvraqdryyssswsewasvpcs

Table 4 also lists, for each amino acid residue of 124C4 listed in Table 4, alternative amino acid residues that could, if present on the same position in 124C4, undergo similar interactions with the corresponding amino acid residues in p19 or p40, respectively, as the amino acid residue that is present at that position in 124C4.

TABLE 4
Binding interactions of Nanobody 124C4 and the p19 or p40 subunit of IL-23

| Amino acid residue | | | | | | Other residues that |
|---|---|---|---|---|---|---|
| in 124C4 | p19 residues | | p40 residues | | | potentially can |
| (numbering | interacting with the | Binding | interacting with the | Binding | Main | undergo similar |
| according to | corresponding residue | energy | corresponding residue | energy | interaction | interactions with p19 |
| Kabat) | in 124C4 | (total) | in 124C4 | (total) | type | or p40 |
| F27 | Q61 | −1.0 | | — | Van der Waals | Y |
| T27b | R64 | −2.0 | | — | Van der Waals, | S, A |
| | | | | | H-bond. | |
| | | | | | electrostatic | |
| D29 | R64, H163, T167 | −3.7 | S245 | −0.9 | Van der Waals, | E, Q, N |
| | | | | | electrostatic | |
| D30 | P60, R64, H163 | −14.0 | P243 | −1.5 | Van der Waals, | E, Q, N |
| | | | | | H-bond, | |
| | | | | | electrostatic | |
| Y31 | Q61 | −1.72 | P243 | −1.0 | Van der Waals | F |
| A32 | | — | T242, P243 | −1.3 | | S, A |
| D51 | | — | T242, P243, H244 | −8.0 | Van der Waals, | E, Q, N |
| | | | | | H-bond, | |
| | | | | | electrostatic | |
| D54 | | — | H244 | −0.8 | Van der Waals | E, Q, N |
| G55 | | — | E235, Y236, H244 | −1.3 | Van der Waals, | A |
| | | | | | electrostatic | |
| S56 | | — | K217, E235, Y236, H244 | −2.0 | Van der Waals | A, T |
| A57 | | — | K217, E235, D238 | −2.8 | Van der Waals, | S |
| | | | | | H-bond, | |
| | | | | | electrostatic | |
| Y58 | | — | K217, D238 | −2.0 | Van der Waals | F, W |
| T97 | D59, P60 | −1.2 | P178, T242, P243, F247 | 2.8 | Van der Waals | S, A |
| G98 | | — | N113, Y114, S115, | −11.1 | Van der Waals, | A |
| | | | P178, S241, T242, F247 | | H-bond, | |
| | | | | | electrostatic | |
| W99 | | — | S115, R117, Q172, | −11.3 | Van der Waals | F, Y |
| | | | W240, S241, T242 | | | |
| G100 | | — | D238, T239, T242 | −2.6 | Van der Waals | A |
| L100a | | — | K217, D238, T239, | −1.1 | Van der Waals | I, M, V |
| | | | T242, H244 | | | |
| N100b | | — | T239 | −0.9 | Van der Waals | D, E, Q |
| Y100f | | — | R117, Q172 | −2.0 | Van der Waals | F, W |

### EXAMPLE 5

### Constructs of the Invention with the 81A12-Based Building Block Towards the N-Terminus

Biparatopic constructs of the invention with the 81A12-based building block towards the N-terminus relative to the 119A3-based building block were made, expressed and compared with a construct with the 119A3-based building block towards the N-end (119A3v16-9GS-Alb8-9GS-81A12v5, which is based on the building blocks 119A3v16 and 81A12v5 as described in WO 09/068,627).

The melting curves of the constructs were determined using DSC at a protein concentration of 0.2 mg/mL in 25 mM Hepes pH 7.5 with 100 mM NaCl, at a heating rate of 1° C./min between 45° C. and 80° C. The constructs with the 81A12-based building block towards the N-terminus (SEQ ID NO's: 21, 22 and 25) gave Tm values of 61.6° C.; 63.8° C. and 64.1° C., respectively, compared to 59.0° C. for the construct with the 119A3-based building block towards the N-terminus.

The potency of the constructs was determined using the mouse splenocyte assay essentially as described in Examples 15 and 25 of WO 09/068,627. The constructs tested were the constructs of SEQ ID NO: 22 and 25. These constructs showed a similar and slightly higher potency (expressed as IC50) of 0.033 nM for SEQ ID NO:22 and 0.039 nM for SEQ ID NO: 25 compared to 0.028 nM for the construct with the 119A3-based building block towards the N-terminus.

The influence of the order of the building blocks on expression levels was determined using a generic high-cell density fermentation process in the Pichia pastoris strain X-33 (Invitrogen). The Ably1 medium, a rich medium containing tryptone as complex component, and standard fermentation parameters such as 30° C., pH5 and 30% dissolved oxygen were used. After the batch phase, a glycerol fed-batch was applied until a Wet Cell Weight of approximately 400 g/L was achieved. Hereafter, induction was started by adding MeOH to the culture. To adapt the culture to MeOH as C-source, an adaptation phase was performed (2 hrs 1.5 mL/L·h followed by 2 hrs at 3 mL/L·h) followed by a constant feed rate of 4 ml/h/L until the end of fermentation (114 hrs total induction time). The fermentation samples were analyzed by RPC analysis, after a proteinA sample clean up, to check for product related variants. Briefly: clarified culture supernatant was mixed with a fixed amount of ProtA resin, and eluted in MQ containing TFA 0.1% and as such are ready for loading on RPC. The constructs with the 81A12-based building blocks towards the N-terminus both gave total concentrations in cell free medium of 1.2 g/L compared to 0.5 g/L for the construct with the 119A3-based building block towards the N-terminus. For the construct of SEQ ID NO: 25, 1.1 g/l, intact material could be obtained and for the construct of SEQ ID NO: 22, 0.8 g/L was obtained. By comparison, only 0.4 g/L intact material could be obtained for the construct with the 119A3-based building block towards the N-terminus.

### EXAMPLE 6

### Construct of the Invention Based on the 37D5 and 124C4 Building Blocks

Melting temperatures, potency and expression levels were determined for the construct of SEQ of the invention based on the 37D5 and 124C4 building blocks, using the same techniques and conditions as used in Example 5. The melting temperature of the construct of SEQ ID NO: 28 was 62.1° C., with a potency in the splenocyte assay of 0.046 nM. Expression levels for SEQ ID NO:28 were comparable to those for the construct 19A3v16-90S-Alb8-9GS-81A12v5 used in Example 5, but lower than for the 81A12/119A3-based constructs with the 81A12-based building block towards the N-terminus.

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

All references disclosed herein are incorporated by reference, in particular for the teaching that is referenced hereinabove.

What is claimed is:

1. Biparatopic protein or polypeptide construct that is specific for and/or directed against IL-23, and that at least comprises:
a) at least one first binding domain, binding unit or binding site that can bind to an epitope of IL-23 that comprises either (i) a stretch of amino acid residues on the p19 subunit of IL-23 that at least comprises the amino acid residues S100, P101 and V102 and/or the amino acid residues F90 and/or P94; and may also comprise comprises the amino acid residues S95, L96 and/or L97: and/or (ii) a stretch of amino acid residues on the p19 subunit of IL-23 that at least comprises the amino acid residues P136, L140, R143 and L147; and that may also comprise the amino acid residues S134 and/or W137; and/or (iii) a stretch of amino acid residues on the p19 subunit of IL-23 that at least comprises the amino acid residues S27 and H29, and that may also comprise the amino acid residues P30. V32, H34 and/or M35; and preferably any two of (i), (ii) and/or (iii) or all of (i), (ii) and/or (iii);
and
b) at least one second binding domain, binding unit or binding site that can bind to an epitope of IL-23 that comprises a stretch of amino acids on the p19 subunit of IL-23 that at least comprises the amino acid residues L85, G86, S87 and/or the amino acid residues F90 and T91 and/or the amino acid residues S95, L96, L97 and P98 and/or the amino acid residues V102, G103, Q104, H106, A107 and/or L110.

2. Biparatopic protein or polypeptide construct according to claim 1, in which
a) the first binding domain is a binding domain, binding unit or binding site that can compete with the Nanobody 119A3 (SEQ ID NO: 1898 in PCT/EP2008/066365) for binding to the epitope defined under a) in claim 1 and/or that can cross-block the binding of 119A3 to the epitope defined under a) in claim 1;
and/or in which
b) the second binding domain is a binding domain, binding unit or binding site that can compete with the Nanobody 81A12 (SEQ ID NO: 1936 in PCT/EP2008/066365) for binding to the epitope defined under b) in claim 1 and/or that can cross-block the binding of 81A12 to the epitope defined under b) in claim 1.

3. Biparatopic protein or polypeptide construct according to claim 1, in which the first and second binding domains are each a domain antibody (or an amino acid sequence that is suitable for use as a domain antibody), a single domain antibody (or an amino acid sequence that is suitable for use as a single domain antibody), a “dAb” (or an amino acid sequence that is suitable for use as a dAb) or a Nanobody®.

4. Biparatopic protein or polypeptide construct according to claim 3, in which
a) the first binding domain is a variant of the Nanobody 119A3 (SEQ ID NO: 1898 in PCT/EP2008/066365);
and/or in which
b) the second binding domain is a variant of the Nanobody 81A12 (SEQ ID NO: 1936 in PCT/EP2008/066365)

5. Biparatopic protein or polypeptide construct according to claim 4, in which the first binding domain is chosen from 119A3v18 (SEQ ID NO:6), 119A3v20 (SEQ ID NO:7), 119A3v21 (SEQ ID NO:8) or 119A3v22 (SEQ ID NO:9).

6. Biparatopic protein or polypeptide construct according to claim 4, in which the second binding domain is chosen from P23IL81A12v5 (SEQ ID NO:2585 in WO 09/068,627) and 81A12v7 (SEQ ID NO: 11).

7. Biparatopic protein or polypeptide construct according to claim 4, in which the second binding domain is towards the N-terminus of the construct compared to the first binding domain.

8. Biparatopic protein or polypeptide construct according to claim 1, which has or has been provided with extended half-life.

9. Biparatopic protein or polypeptide construct that is specific for and/or directed against IL-23, and that at least comprises:
a) at least one first binding domain, binding unit or binding site that can bind to an epitope of IL-23 that comprises either (i) a stretch of amino acid residues on the p19 subunit of IL-23 that at least comprises the amino acid residues K20, T23 and L24 and/or the amino acid residues W26, S27, A28 and H29; and/or (ii) a stretch of amino acid residues on the p19 subunit of IL-23 that at least comprises the amino acid residues E93, P94, S95, L96, L97, P98, D99, S100, P101 and/or V102; and/or (iii) a stretch of amino acid residues on the p19 subunit of IL-23 that at least comprises the amino acid residues W137, L140 and/or R143; and preferably any two of (i), (ii) and/or (iii) or all of (i), (ii) and/or (iii);
and
b) at least one second binding domain, binding unit or binding site that can bind to the p19/p40 interface of IL-23, and in particular to an epitope of IL-23 that comprises either (i) a stretch of amino acids on the p19 subunit of IL-23 that at least comprises the amino acid residue H163 and optionally also the amino acid residue T167; and/or (ii) stretch of amino acids on the p40 subunit of IL-23 that at least comprises the amino acid residues W240, S241, T242, H244 and/or F247; and/or (iii) a stretch of on the p40 subunit of IL-23 that at least comprises the amino acid residues N113, Y114, S115 and/or R117; and preferably any two of (i), (ii) and/or (iii) or all of (i), (ii) and/or (iii).

10. Biparatopic protein or polypeptide construct according to claim 9, in which
a) the first binding domain is a binding domain, binding unit or binding site that can compete with the Nanobody 37D5 (SEQ ID NO: 2490 in PCT/EP2008/066365) for binding to the epitope defined under a) in claim 9 and/or that can cross-block the binding of 37D5 to the epitope defined under a) in claim 9;
and/or in which
b) the second binding domain is a binding domain, binding unit or binding site that can compete with the Nanobody 124C4 (SEQ ID NO: 1932 in PCT/EP2008/066365) for binding to the epitope defined under b) in claim 9 and/or that can cross-block the binding of 124C4 to the epitope defined under b) in claim 9.

11. Biparatopic protein or polypeptide construct according to claim 9, in which the first and second binding domains are each a domain antibody (or an amino acid sequence that is suitable for use as a domain antibody), a single domain antibody (or an amino acid sequence that is suitable for use as a single domain antibody), a “dAb” (or an amino acid sequence that is suitable for use as a dAb) or a Nanobody®.

12. Biparatopic protein or polypeptide construct according to claim 11, in which
a) the first binding domain is a variant of the Nanobody 37D5 (SEQ ID NO: 2490 in PCT/EP2008/066365);
and/or in which
b) the second binding domain is a variant of the Nanobody 124C4 (SEQ ID NO: 1932 in PCT/EP2008/066365).

13. Biparatopic protein or polypeptide construct according to claim 12, in which the first binding domain is chosen from P23IL37D5V17 (SEQ ID NO: 2602 in WO 09/068,627) or 37D5v18 (SEQ ID NO:15).

14. Biparatopic protein or polypeptide construct according to claim 12, in which the second binding domain is chosen from 124C4v5 (SEQ ID NO: 12). 124C4v6 (SEQ ID NO: 13) or 124C4v7 (SEQ ID NO: 14).

15. Biparatopic protein or polypeptide construct according to claim 9, which has or has been provided with extended half-life.
